(19) United States
(12) Patent Application Publication
(10) Pub. No.: US 2025/0235192 A1
(43) Pub. Date: Jul. 24, 2025

(54) AORTIC LEAFLET CROSSING SYSTEMS

(71) Applicant: Medtronic Vascular, Inc., Santa Rosa, CA (US)

(72) Inventors: Timothy D. FARRELL, Galway (IE); Niall F. DUFFY, Galway (IE); Constantin F. CIOBANU, Galway (IE); David J. OTOOLE, Galway (IE); Evelyn C. COLLIER, Galway (IE)

(21) Appl. No.: 19/032,641

(22) Filed: Jan. 21, 2025

Related U.S. Application Data
(60) Provisional application No. 63/624,013, filed on Jan. 23, 2024.

(51) Int. Cl. A61B 17/00

(52) U.S. Cl. CPC A61B 17/00234; A61B 2017/00243; A61B 2017/00305; A61B 2017/00367; A61B 2017/00477

(57) ABSTRACT

An aortic leaflet crossing system includes a guide catheter defining a guide lumen therein. The guide catheter has a proximal end defining a proximal opening and a distal end defining a distal opening. The aortic leaflet crossing system further includes a plurality of guidewires extending into the proximal opening and through the guide lumen and out of the distal opening. The aortic leaflet crossing system also includes a control device configured to advance the plurality of guidewires such that at least one of the guidewires crosses aortic leaflets of an aortic valve and to retract guidewires in the plurality of guidewires that do not cross the aortic leaflets.

### CROSS-REFERENCE TO RELATED APPLICATIONS

This application claims the benefit of U.S. Provisional Patent Application Ser. No. 63/624,013, filed Jan. 23, 2024, the entire content of which is incorporated herein by reference.

### TECHNICAL FIELD

The present application is generally related to aortic leaflet crossing systems including multiple guidewires to provide a system where one or more guidewires cross the aortic leaflets.

### BACKGROUND

Prosthetic heart valve designs aim to mimic the function of natural heart valve designs. One type of prosthetic heart valve design is referred to as a self-expanding valve (e.g., a supra-annular design). The self-expanding valve design includes an expandable stent frame formed of a self-expanding material (e.g., a nickel titanium alloy or Nitinol). Prosthetic leaflets may be mounted onto the expandable stent frame to replicate natural aortic leaflets.

Prosthetic heart valve delivery systems are delivered using procedures which may utilize several guidewires and catheters to provide a guided path for delivery of the prosthetic heart valve to an implementation site. This guided path extends through the aortic leaflets and typically includes guidewires and catheters extending through the opening defined by the aortic leaflets. Stenosed aortic leaflets do not open as much as healthy aortic leaflets. Therefore, applying the prosthetic heart valve delivery procedure to stenosed aortic leaflets may be challenging due to the difficulty of crossing the stenosed aortic leaflets with guidewires and catheters.

### SUMMARY

In a first embodiment, an aortic leaflet crossing system is disclosed. The aortic leaflet crossing system may include a guide catheter defining a guide lumen therein. The guide catheter may include a proximal end defining a proximal opening and a distal end defining a distal opening. The aortic leaflet crossing system further includes a plurality of guidewires extending into the proximal opening and through the guide lumen and out of the distal opening. The aortic leaflet crossing system may further include a control device (e.g., a control knob) configured to advance the plurality of guidewires such that at least one of the guidewires crosses aortic leaflets of an aortic valve and to retract guidewires in the plurality of guidewires that do not cross the aortic leaflets.

The control device may be configured to translate axially along a longitudinal axis of the guide catheter to advance the plurality of guidewires. The control device may define a plurality of apertures where the plurality of guidewires is connected to the control device through the plurality of apertures. The control device may include a proximal surface and a distal surface. The plurality of apertures may extend through the proximal surface and the distal surface. In one or more embodiments, a first group of the plurality of apertures is arranged in a first concentric region of the control device and a second group of the plurality of apertures are arranged in a second concentric region of the control device. The second concentric region may have a second diameter greater than a first diameter of the first concentric region. The second group may contain more guidewires than the first group. The plurality of guidewires may include 15 or more guidewires.

In a second embodiment, an aortic leaflet crossing system is disclosed. The aortic leaflet crossing system includes a guide catheter defining a plurality of guide lumens having a plurality of proximal ends defining a plurality of proximal openings and a plurality of distal ends defining a plurality of distal openings. The aortic leaflet crossing system further includes a plurality of guidewires extending into the plurality of proximal openings and through the plurality of guide lumens and out of the plurality of distal openings. The aortic leaflet crossing system further includes a control device configured to advance the plurality of guidewires such that at least one of the guidewires crosses aortic leaflets of an aortic valve and to retract guidewires in the plurality of guidewires that do not cross the aortic leaflets.

In one or more embodiments, one of the plurality of guide lumens is aligned with a longitudinal axis of the guide catheter. A first group of the plurality of guide lumens may be arranged in a first concentric region of the guide catheter. In one or more embodiments, the guide catheter includes a cylindrical portion and a tapered portion distally extending from the cylindrical portion, where the tapered portion tapers outward from a proximal end to a distal end. The plurality of guide lumens in the cylindrical portion may have a first average spacing, the plurality of guide lumens in the tapered portion may have a second average spacing, and the second average spacing may be greater than the first average spacing. In one or more embodiments, at least some of the guide lumens are angled relative to a longitudinal axis of the guide catheter. In one or more embodiments, at least one of the guide lumens is aligned with a longitudinal axis of the guide catheter. The plurality of guide lumens may include 5 or more guide lumens, and the plurality of guidewires may include 5 or more guidewires.

In another embodiment, an aortic leaflet crossing method is disclosed. The aortic leaflet crossing method includes inserting a guide catheter carrying a plurality of guidewires into a vasculature. The guidewire has a proximal end defining a proximal opening and a distal end defining a distal opening. The method further includes delivering the guide catheter to an aorta. The method also includes advancing the plurality of guidewires through the guide catheter and out of the distal opening such that at least one of the guidewires crosses aortic leaflets of an aortic valve. The method further includes retracting guidewires in the plurality of guidewires that do not cross aortic leaflets of the aortic valves.

The retracted guidewires may be obstructed by the aortic leaflets. The retracted guidewires and the guide catheter may be removed from the vasculature of the patient as part of the method. The method may further include advancing a second guide catheter over one of the at least one guidewire crossing the aortic leaflets of the aortic valve. The retracted guidewires may be withdrawn through a control device configured to advance and to retract the guidewires.

### BRIEF DESCRIPTION OF THE DRAWINGS

FIG. 1A depicts a cross-sectional view of a patient anatomy and a schematic, side view of a pre-curved guidewire extending through the patient anatomy.

FIG. 1B depicts a cross-sectional view of the patient anatomy and a schematic, side view of a delivery system tracking over the pre-curved guidewire in a pre-deployment state.

FIG. 1C depicts a cross-sectional view of the patient anatomy and a schematic, side view of a transcatheter aortic valve replacement (TAVR) device in a partially deployed position.

FIG. 1D depicts a cross-sectional view of the patient anatomy and a schematic, side view of the TAVR device in a fully deployed position.

FIG. 2A depicts a cross-sectional view of a patient anatomy and a schematic, side view of a guide catheter extending through the patient anatomy.

FIG. 2B depicts a top view of a stenosed valve in an open state.

FIG. 2C depicts a top view of a stenosed valve in a closed state.

FIG. 2D depicts a top view of a healthy valve in an open state.

FIG. 2E depicts a top view of a healthy valve in a closed state.

FIG. 3A depicts a schematic, side view of multiple guidewires carried in an aortic leaflet crossing system guide catheter shown in cross section.

FIG. 3B depicts a schematic, side view of the multiple guidewires in a first deployment position in which a guidewire has crossed (e.g., extended beyond) aortic leaflets.

FIG. 3C depicts a cross section view taken at line 3C-3C of FIGS. 3A and 3B thereby showing the orientation and spacing of multiple guidewires.

FIG. 4A depicts a side view of an aortic leaflet crossing system including multiple guidewires carried in a guide catheter.

FIG. 4B depicts a cross section view taken at line 4B-4B of FIG. 4A thereby showing the orientation and spacing of the multiple lumens.

FIG. 5A depicts a side view of an aortic leaflet crossing system including multiple guidewires carried in a guide catheter.

FIG. 5B depicts a cross section view taken at line 5B-5B of FIG. 5A thereby showing the orientation and spacing of the multiple lumens at a first longitudinal cross section of the guide catheter.

FIG. 5C depicts a cross section view at line 5C-5C of FIG. 5A thereby showing the orientation and spacing of the multiple lumens at a second longitudinal cross section of the guide catheter.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

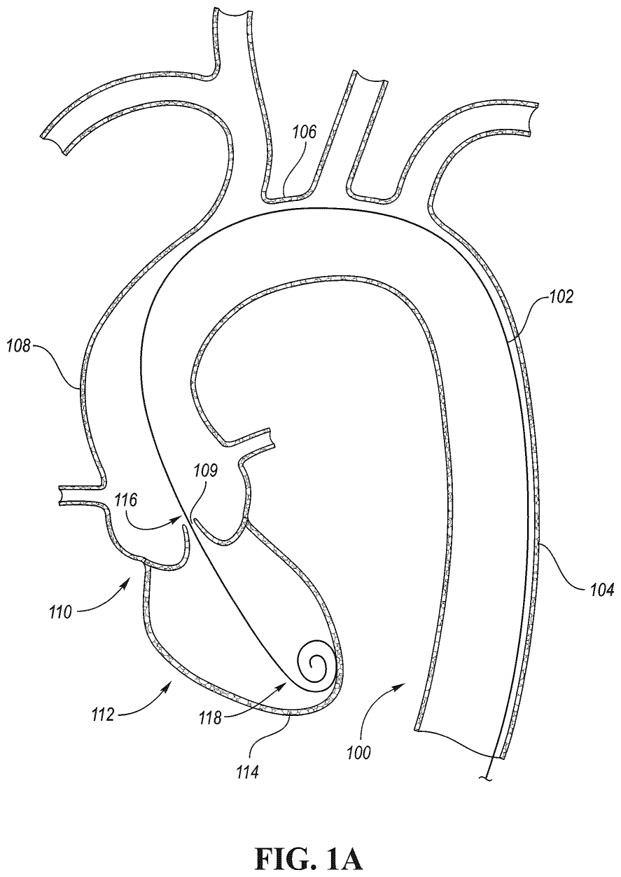

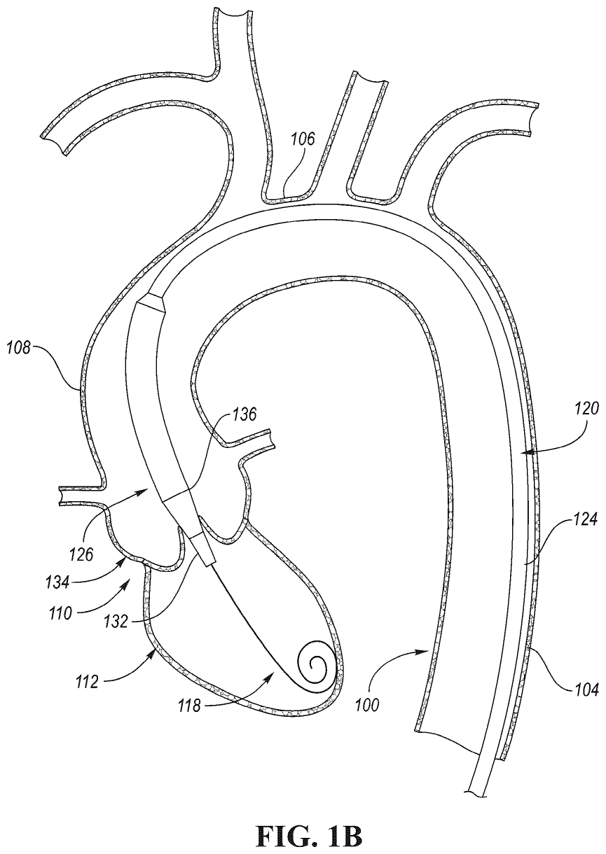

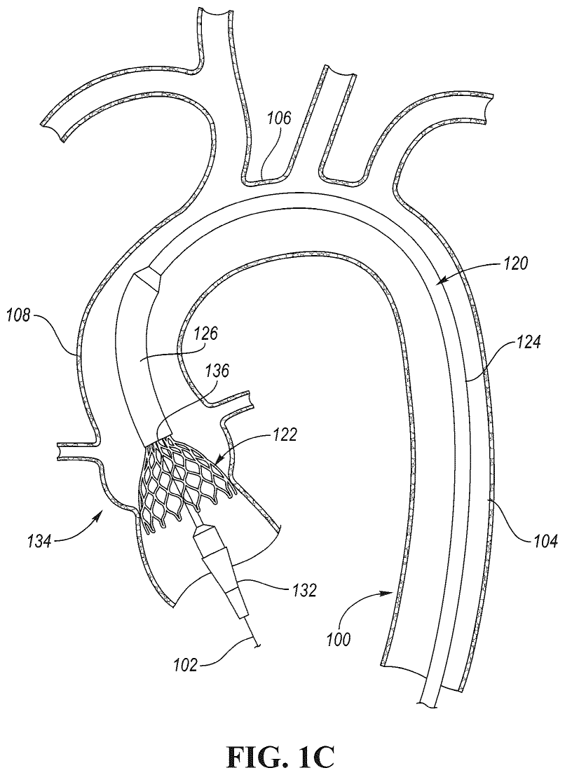

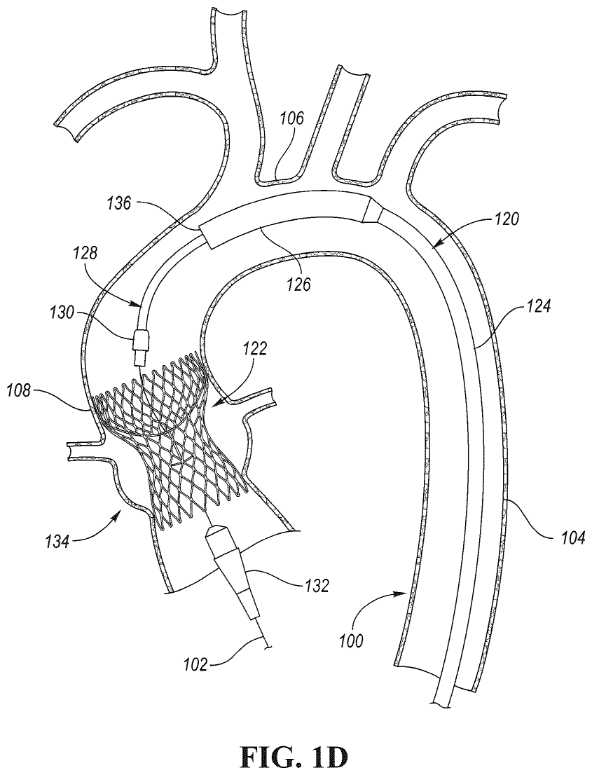

FIG. 1A depicts a cross-sectional view of patient anatomy 100 and a schematic, side view of pre-curved guidewire 102 extending through patient anatomy 100. Patient anatomy 100 includes descending aorta 104, aortic arch 106, ascending aorta 108, aortic leaflets 110, and heart 112 having heart wall 114. Descending aorta 104 is in communication with aortic arch 106. Aortic arch 106 is in communication with ascending aorta 108. Ascending aorta 108 is in communication with aortic leaflets 110 of an aortic valve.

Aortic leaflets 110 may be native aortic leaflets or replacement, prosthetic aortic leaflets. The replacement aortic leaflets may be a surgical aortic valve replacement (SAVR) device or a transcatheter aortic valve replacement (TAVR) device, such as a self-expanding TAVR device or a balloon inflatable TAVR device. Such devices generally include a frame (e.g., a stent frame) and a prosthetic valve. A TAVR device may be delivered to an implantation site using pre-curved guidewire 102 to track the TAVR device in a delivery state around patient anatomy 100 and through aortic leaflets 110.

Pre-curved guidewire 102 includes substantially straight portion 116 and pre-curved portion 118 extending from substantially straight portion 116. In one or more embodiments, pre- curve portion 118 forms a flexible tip region having greater flexibility than substantially straight portion 116, which may be considered a stiff region. Pre-curved portion may be a self-expanding structure formed of a metal alloy (e.g., stainless steel alloy, nickel titanium alloy, Nitinol, or other shape memory material).

As shown in FIG. 1A, pre-curved guidewire 102 extends through opening 109 defined by aortic leaflets 110 in a delivery position. The delivery position may be achieved using the following procedure. First, a straight tip guidewire is inserted into a patient's vasculature and advanced through opening 109 of aortic leaflets 110. Subsequently, a catheter (e.g., angiographic catheter) is placed over the straight tip guidewire and is advanced into heart 112 (e.g., a ventricle of heart 112). The straight tip guidewire is then removed from the catheter and the patient's vasculature, and replaced with a J-tip wire, which is inserted into the patient's vasculature and advanced through the catheter. The catheter may then be exchanged for a second catheter (e.g., pigtail catheter) by withdrawing the catheter and then inserting the second catheter into the patient's vasculature and advancing it over the catheter-tip wire. The J-tip wire is then exchanged for pre-curved guidewire 102 by advancement through the second catheter. While pre-curved guidewire 102 advances through the second catheter, pre-curved portion 118 is straightened by the second catheter in a constrained state. When pre-curved portion 118 emerges from the distal end of the second catheter, pre-curved portion 118 takes on its pre-curved shape as shown in FIG. 1A and may contact heart wall 114. The above process for establishing a curved guidewire in the left ventricle is merely an example, and other steps or devices may be used to establish the guidewire.

FIG. 1B depicts a cross-sectional view of patient anatomy 100 and a schematic, side view of delivery system 120 tracking over pre-curved guidewire 102 in a pre-deployment state. FIG. 1C depicts a cross-sectional view of patient anatomy 100 and a schematic, side view of delivery system 120 and TAVR device 122 in a partially deployed position. FIG. 1D depicts a cross-sectional view of patient anatomy 100 and a schematic, side view of delivery system 120 and TAVR device 122 in a fully deployed position.

Delivery system 120 includes delivery sheath assembly 124, capsule 126, inner shaft assembly 128, retention hub 130, and tip 132. Delivery system 120 is configured to retain TAVR device 122 in a delivery state in which TAVR device 122 is loaded in a constrained position within delivery system 120. In the delivery state, TAVR device 122 is coupled to inner shaft assembly 128 via retention hub 130 (e.g., TAVR device 122 may include eyelets captured by retention hub 130) and is compressively retained within capsule 126 of delivery sheath assembly 124. Loaded delivery system 120 may be configured to percutaneously deliver TAVR device 122 to implantation site 134 (e.g., a defective heart valve). As shown in FIGS. 1C and 1D, loaded delivery system 120 may be advanced toward implantation site over pre-curved guidewire 102 in a retrograde manner through the patient's femoral artery into descending aorta 104 over aortic arch 106 through ascending aorta 108 and across a defective heart valve (e.g., about mid-way through defective heart valve) at implantation site 134.

For self-expanding embodiments, delivery sheath assembly 124 is configured to withdraw capsule 126 proximally from TAVR device 122 via operation of a handle (not shown) on delivery system 120, thereby allowing TAVR device 122 to expand from the constrained, pre-deployment position to a partially deployed position shown in FIG. 1C and fully deployed position shown in FIG. 1D in which TAVR device 122 is fully released from capsule 126 and TAVR device 122 is implanted to the native valve. A release sheath assembly of delivery system 120 may be configured to fully release TAVR device 122 from capsule 126. The handle may be configured to maneuver capsule 126 to the partially deployed position shown in FIG. 1C in which a distal region of TAVR device 122 is permitted to self-expand at distal end 136 of retention hub 130 while a proximal region of TAVR device 122 remains coupled to retention hub 130.

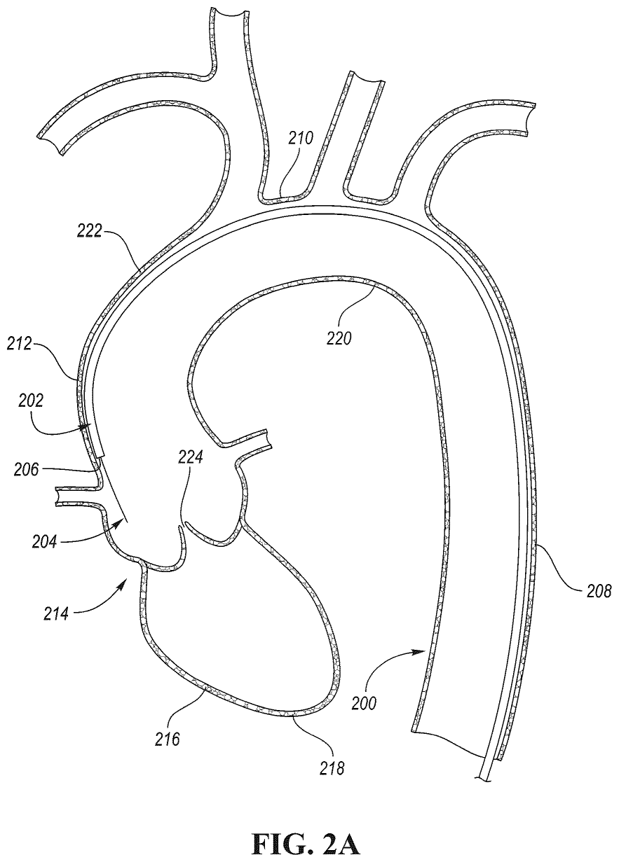

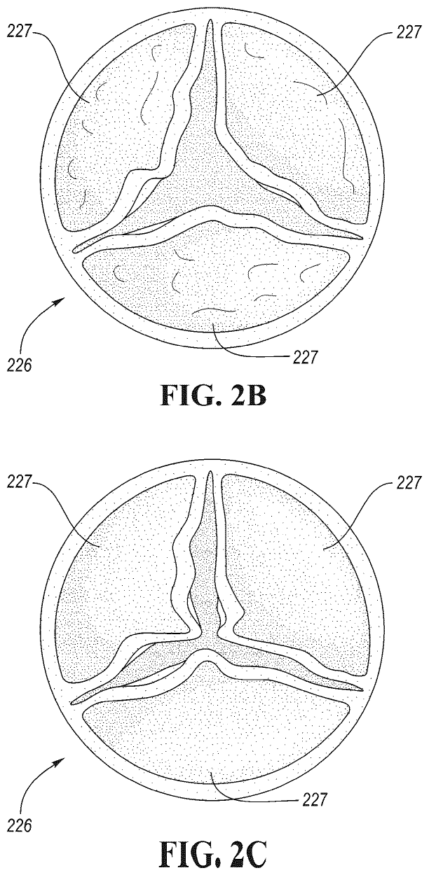

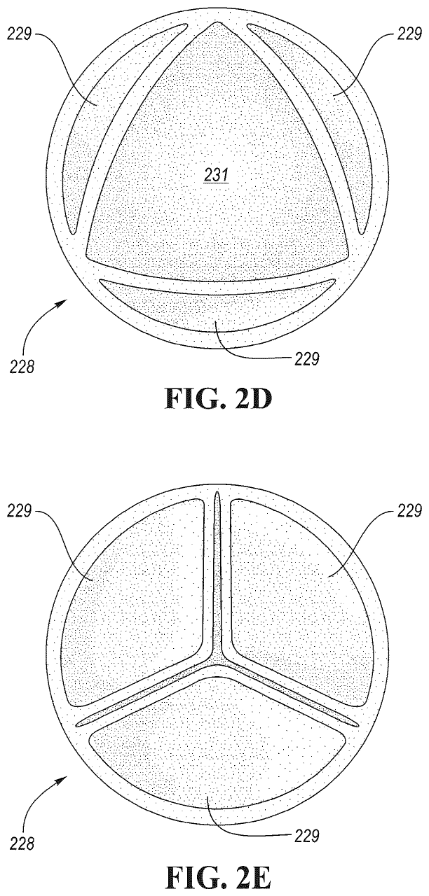

FIG. 2A depicts a cross-sectional view of patient anatomy 200 and a schematic, side view of guide catheter 202 extending through patient anatomy 200. As shown in FIG. 2A, straight tip guidewire 204 has extended through distal end 206 of guide catheter 202. Patient anatomy 200 includes descending aorta 208, aortic arch 210, ascending aorta 212, aortic leaflets 214, and heart 216 having heart wall 218. The aorta includes outer curve 222 and inner curve 220.

As shown in FIG. 2A, guide catheter 202 and straight tip guidewire 204 has aligned with outer curve 222 of the aorta. This alignment may occur due to the stiffness of the body of guide catheter 202 and straight tip guidewire 204 causing these components to bow outward toward outer curve 222. As shown in FIG. 2A, guide catheter 202 and straight tip guidewire 204 are not aligned with opening 224 defined by aortic leaflets 214. This misalignment may present a challenge to deployment of a TAVR device at an implantation site.

While misalignment may increase the amount of time taken to cross opening 224, one or more other factors may increase implantation procedure time. FIG. 2B depicts a top view of stenosed valve 226 in an open state. FIG. 2C depicts a top view of stenosed valve 226 in a closed state. Stenosed valve 226 includes stenosed aortic leaflets 227. FIG. 2D depicts a top view of healthy valve 228 in an open state. FIG. 2E depicts a top view of healthy valve 228 in a closed state. Healthy valve 228 includes healthy aortic leaflets 229. In healthy valve 228, blood flows out of heart 216 through opening 231 in the open state while heart 216 beats and closes substantially in the closed state. In the open state of healthy valve 228, opening 231 provides a large opening for crossing the annulus, although the force of blood flowing out of heart 216 works against catheter(s) and/or guidewire(s) crossing the annulus. However, crossing the annulus in the closed state with healthy valve 228 substantially closed presents difficulties. The difficulties are present in both the open and closed states of stenosed valve 226, where the stenosed aortic leaflets 227 do not fully open or close but are rather in a partially closed state. Another factor that may contribute to the difficulty in crossing the annulus is that the catheter(s) and/or guidewire(s) may get caught on the aortic leaflets. These difficulties may add to procedure time where reducing procedure time may enhance the benefit of the procedure.

Considering the foregoing, what is needed is an aortic leaflet crossing system to address one or more of the factors identified above that may contribute to increased TAVR device implantation procedure time. In one or more embodiments, aortic leaflet crossing systems are disclosed that may repeatedly and consistently cross aortic leaflets with a guidewire while reducing the instance of one or more of the challenging factors identified above.

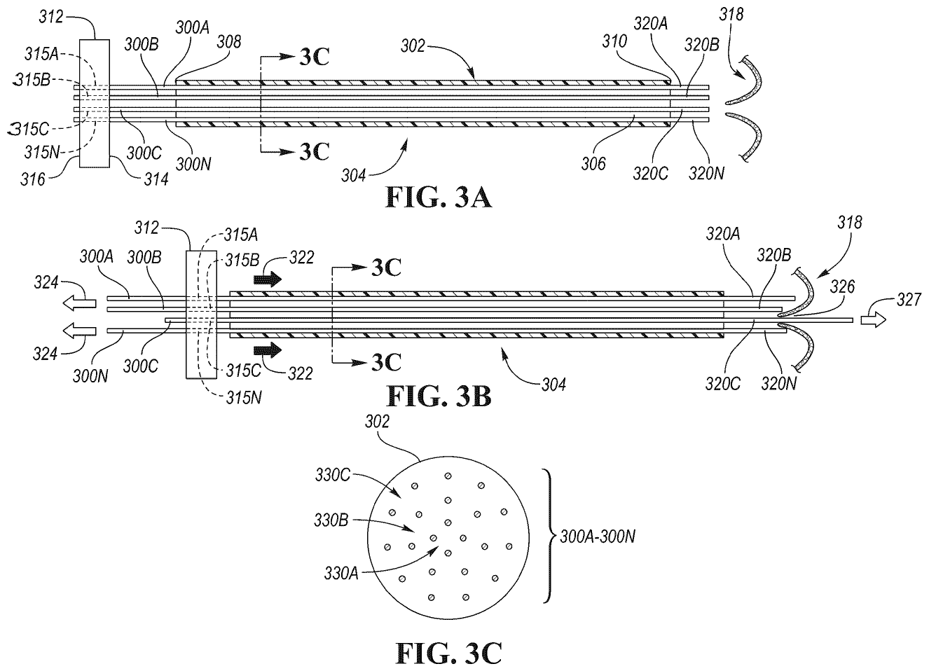

FIG. 3A depicts a schematic, side view of multiple guidewires 300A, 300B, 300C, and 300N carried in guide catheter 302 (shown in cross section) of aortic leaflet crossing system 304. Guide catheter 302 defines single lumen 306 for carrying multiple guidewires 300A, 300B, 300C, and 300N. Multiple guidewires 300A, 300B, 300C, and 300N are configured to extend from proximal end 308 of guide catheter 302 through distal end 310 of guide catheter 302. While four (4) guidewires are shown in FIG. 3A, more than four (4) guidewires may be carried within single lumen 306 of guide catheter 302 depending on the diameter of guide catheter 302.

In one embodiment, when the outer diameter of guide catheter 302 may be 12 French, single lumen 306 may accommodate up to about 90 guidewires having a diameter of 0.014 inches and up to 18 guidewires having a diameter of 0.036 inches. In another embodiment, when the outer diameter of guide catheter 302 is 4 French, single lumen 306 may accommodate up to 16 guidewires having a diameter of 0.014 inches. any of the following number of guidewires or any range including any two of the following number of guidewires: 5, 6, 7, 8, 9, 10, 11, 12, 13, 14, 15, 16, 17, 18, 19, and 20. Increasing the number of guidewires may increase the likelihood that one or more of the guidewires cross the aortic leaflets. In one or more embodiments, one or more of the guidewires have a diameter of 0.15 millimeters. One or more of the guidewires may have any of the following diameters or a range of any two of the following diameters: 0.10, 0.11, 0.12, 0.13, 0.14, 0.15, 0.16, 0.17, 0.18, 0.19, and 0.20 millimeters.

Aortic leaflet crossing system 304 further includes control knob 312. Control knob 312 defines multiple apertures 315A, 315B, 315C, and 315N that extend from distal surface 314 to proximal surface 316 of control knob 312. Multiple guidewires 300A, 300B, 300C, and 300N are connected through corresponding apertures in control knob 312 at proximal portions of guidewires 300A, 300B, 300C, and 300N. In one or more embodiments, the multiple guidewires are loosely connected through the apertures of control knob 312 such that a force exerted on control knob 312 in a longitudinal direction of guide catheter 302 advances the multiple guidewires in the longitudinal direction while an opposing force (e.g., a reaction force or a pulling force) may retract one or more of the multiple guidewires. A reaction force may be exerted on one or more of the guidewires when one or more of the guidewires contacts the aortic wall or an aortic leaflet, thereby causing the one or more guidewires to retract (e.g., push backwards) due to the reaction force. A pulling force may be exerted by a clinician by grasping a proximal portion of one or more of the guidewires. The pulling force may be used on the guidewires that do not cross the aortic leaflets.

While FIG. 3A depicts aortic leaflet crossing system 304 in a delivery position, FIG. 3B depicts aortic leaflet crossing system 304 in a first deployment position in which guidewire 300C has crossed (e.g., extended beyond) aortic leaflets 318. In FIG. 3A, distal ends 320A, 320B, 320C, and 320N of guidewires 300A, 300B, 300C, and 300N, respectively, are approaching but are proximally spaced apart from aortic leaflets 318. As shown by arrows 322 in FIG. 3B, control knob 312 is advanced in a longitudinal, distal direction, thereby advancing guidewires 300A, 300B, 300C, and 300N toward aortic leaflets 318 until distal ends 320A, 320B, and 320N of guidewires 300A, 300B, and 300N contact aortic leaflets 318 and push backwards due to a reaction force as shown by arrows 324. As shown in FIG. 3A, all the guidewires are pushed through the guide catheter together. In other embodiments, the guidewires may be pushed through in groups or sequentially.

As shown in FIG. 3B, guidewire 300C is aligned with opening 326 defined by aortic leaflets 318, thereby extending therethrough. Due to the reaction force, guidewires 300A, 300B, and 300N extend proximally a longer distance than guidewire 300C, which extends through opening 326 of aortic leaflets 318 to maintain its extension length beyond control knob 312 in the proximal direction. The difference in lengths between guidewires 300A, 300B, and 300N and guidewire 300C signals to the clinician that guidewire 300C has crossed aortic leaflets 318, as depicted by arrow 327. While FIG. 3B depicts one guidewire (i.e., guidewire 300C) crossing aortic leaflets 318, in other embodiments, more than one guidewire may cross aortic leaflets 318. At this point in the procedure, the guidewires that did not cross aortic leaflets 318 may be removed from guide catheter 302 to provide more space for subsequent procedural steps (e.g., inserting a guide catheter over guidewire 300C and exchanging it for a pigtail and/or J-tip wire). In one or more embodiments, guide catheter 302 may be removed from the patient's vasculature before these subsequent steps and optionally replaced with a different catheter.

FIG. 3C depicts a cross section view taken at line 3C-3C shown in FIGS. 3A and 3B thereby showing the orientation and spacing of multiple guidewires 300A to 300N. As shown in FIG. 3C, guidewires 300A to 300N are generally concentrically arranged in first, second and third concentric regions 330A, 330B, and 330C. As shown in FIG. 3C, first concentric region 330A includes four (4) guidewires, second concentric region 330B includes seven (7) guidewires, and third concentric region 330C includes eleven (11) guidewires, for a total of twenty-two (22) guidewires. The total number of guidewires may vary depending on the diameter of guide catheter 302. The concentric arrangement of a greater number of guidewires in larger concentric regions is configured to maintain spacing between the guidewires such that a large cross-sectional area is covered with guidewires so that at least one guidewire crosses the aortic leaflets. In one or more embodiments, the apertures of the control knob 312 are fixed such that the apertures contribute to the spacing of the guidewires within guidewire lumen 306. As the guidewires extend more distally toward the aortic leaflets, the guidewire locations may become commingled but the resilient and flexible characteristics of the guidewires may aid in maintaining cross sectional coverage by the guidewires.

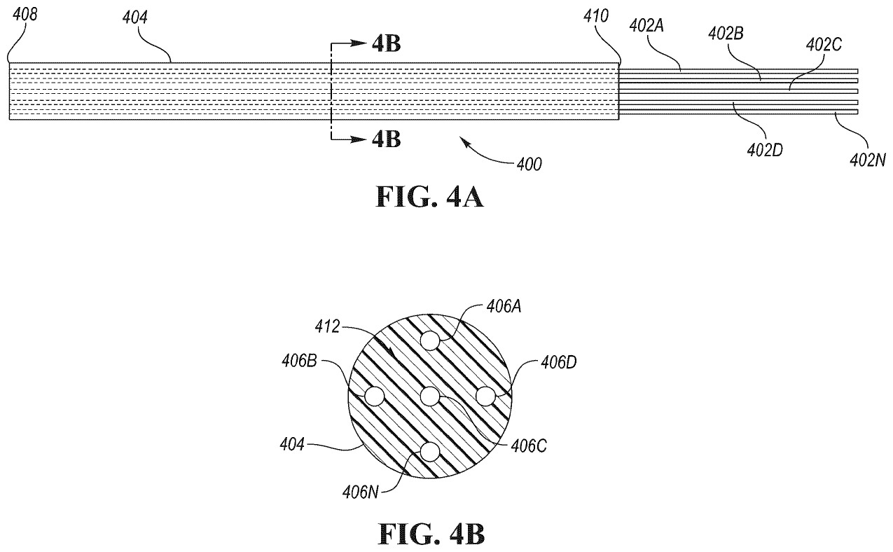

FIG. 4A depicts a side view of aortic leaflet crossing system 400 including multiple guidewires 402A, 402B, 402C, 402D, and 402N carried in guide catheter 404. FIG. 4B depicts a cross section view taken at line 4B-4B of FIG. 4A thereby showing the orientation and spacing of multiple lumens 406A to 406N for receiving multiple guidewires 402A to 402N. Lumens 406A, 406B, 406C, 406D, and 406N longitudinally extend the length of guide catheter 404 such that guidewires 402A, 402B, 402C, 402D, and 402N may enter proximal end 408 of guide catheter 404 and extend through distal end 410 of guide catheter 404. In one embodiment, the diameter of lumens 406A, 406B, 406C, 406D, and 406N are 0.90 millimeters. In other embodiments, the diameter of lumens 406A, 406B, 406C, 406D, and 406N may be any of the following values or in a range of any two of the following values: 0.80, 0.85, 0.90, 0.95, and 1.0 millimeters.

As shown in FIG. 4B, lumen 406C is located along the longitudinal axis of guide catheter 404 and lumens 406A, 406B, 406D, and 406N are arranged within concentric region 412 of guide catheter 404 such that all the lumens are spaced apart from each other. The average spacing of lumens 406A, 406B, 406D, and 406N from 406C may be any of the following values or in a range of any two of the following values: 0.80, 0.85, 0.90, 0.95, and 1.0 millimeters. The average spacing between lumens 406A, 406B, 406D, and 406N may be any of the following values or in a range of any two of the following values: 1.6, 1.7, 1.8, 1.9, and 2.0 millimeters. In one or more embodiments, the average radial spacing (e.g., the spacing of lumens 406A, 406B, 406D, and 406N from 406C) is less than the average concentric spacing (e.g., the spacing between lumens 406A, 406B, 406D, and 406N). The spacing of lumens are configured to maintain the spacing of guidewires 402A, 402B, 402C, 402D, and 402N within guidewire catheter 404 and to aid in coverage of a cross section area corresponding to the aortic leaflets such that at least one of the guidewires crosses the aortic leaflets.

While five (5) guidewire/lumen combinations are shown in FIGS. 4A and 4B, more than five (5) guidewirc/lumen combinations may be carried within guide catheter 404 (e.g., 6, 7, 8, 9, 10, 11, and 12 guidewire/lumen combinations). In one embodiment, the outer diameter of guide catheter 404 may be 18 French. When the outer diameter of guide catheter 404 is 18 French, guide catheter 404 may accommodate any of the following number of guidewires or any range including any two of the following number of guidewires: 4, 5, 6, 7, 8, 9, 10, 11, and 12. Increasing the number of guidewires may increase the likelihood that one or more of the guidewires cross the aortic leaflets. In other embodiments, guide catheter 404 may have an outer diameter of any of the following or in a range of any two of the following: 15, 16, 17, 18, 19, 20, and 21 French. In one or more embodiments, the number of guidewires for any suitable guide catheter outer diameter may be any of the following or in a range of any two of the following: 4, 5, 6, 7, 8, 9, 10, 11, 12, 13, 14, 15, 16, 17, 18, 19, and 20. In one or more embodiments, one or more of the guidewires have a diameter of 0.89 millimeters. One or more of the guidewires may have any of the following diameters or a range of any two of the following diameters: 0.75, 0.80, 0.85, 0.89, 0.95, and 1.0 millimeters.

In one or more embodiments, multiple guidewires 402A, 402B, 402C, 402D, and 402N are loosely connected through the apertures of a control knob such that a force exerted on control knob in a longitudinal direction of guide catheter 404 advances the multiple guidewires in the longitudinal direction while an opposing force (e.g., a reaction force or a pulling force) may retract one or more of the multiple guidewires. A reaction force may be exerted on one or more of the guidewires when one or more of the guidewires contacts the aortic wall or an aortic leaflet, thereby causing the one or more guidewires to retract (e.g., push backwards) due to the reaction force. A pulling force may be exerted by a clinician by grasping a proximal portion of one or more of the guidewires. The pulling force may be used on the guidewires that do not cross the aortic leaflets to remove those guidewires before subsequent procedural steps (e.g., inserting a guide catheter over one of the crossing guidewires and exchanging it for a pigtail and/or J-tip wire). In one or more embodiments, guide catheter 404 may be removed from the patient's vasculature before these subsequent steps.

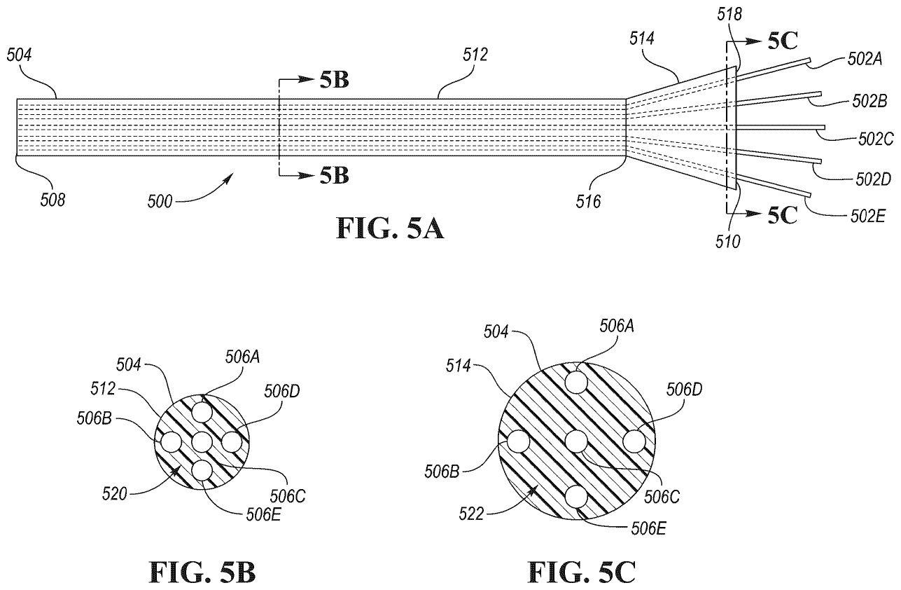

FIG. 5A depicts a side view of aortic leaflet crossing system 500 including multiple guidewires 502A, 502B, 502C, 502D, and 502N carried in guide catheter 504. FIG. 5B depicts a cross section view taken at line 5B-5B of FIG. 5A thereby showing the orientation and spacing of multiple guidewires 502A through 502N at a first longitudinal cross section of guide catheter 504. FIG. 5C depicts a cross section view at line 5C-5C of FIG. 5A thereby showing the orientation and spacing of multiple guidewires 502A through 502N at a second longitudinal cross section of guide catheter 504.

Multiple guidewires 502A, 502B, 502C, 502D, and 502N extend within lumens 506A, 506B, 506C, 506D, and 506N, respectively, defined by guide catheter 504. Lumens 506A, 506B, 506C, 506D, and 506N longitudinally extend the length of guide catheter 504 such that guidewires 502A, 502B, 502C, 502D, and 502N may enter proximal end 508 of guide catheter 504 and extend through distal end 510 of guide catheter 504. In one embodiment, the diameter of lumens 506A, 506B, 506C, 506D, and 506N are 0.90 millimeters. In other embodiments, the diameter of lumens 506A, 506B, 506C, 506D, and 506N may be any of the following values or in a range of any two of the following values: 0.80, 0.85, 0.90, 0.95, and 1.0 millimeters.

As shown in FIG. 5A, guide catheter 504 includes cylindrical portion 512 and tapered portion 514 extending from cylindrical portion 512. Tapered portion 514 tapers outward from proximal end 516 to distal end 518. In one embodiment, the outer diameter of cylindrical portion 512 may be 12 French while the outer diameter of tapered portion 514 flares out to 18 French at distal end 518. In one or more embodiments, the outer diameter of distal end 518 of tapered portion 514 is greater than the outer diameter of cylindrical portion 512. In one or more embodiments, the smaller and larger diameters may be selected from the following diameters: 10, 11, 12, 13, 14, 15, 16, 17, 18, 19, and 20 French.

Lumens 506A, 506B, 506C, 506D, and 506N follow the taper of tapered portion 514 such that the lumens are spaced out further from each other at distal end 518 than in cylindrical portion 512. By flaring out the outer diameter of guide catheter 504, the guidewires are spaced further apart from each outer and/or angled outward relative to the longitudinal axis of guide catheter 504 (e.g., guidewires 502A, 502B, 502D, and 502N are angled, guidewire 502C follows the longitudinal axis of guide catheter 504, and the angling spaces out guidewires 502A, 502B, 502D, and 502N). The angling may be any of the following angles or in a range of any two of the following angles: 15, 20, 25, 30, 35, and 40 degrees. The increased spacing is configured to provide a spreading of guidewires to provide a likelihood of one of the wires entering the aortic leaflets.

As shown in FIG. 5B, lumen 506C is located along the longitudinal axis of guide catheter 504 and lumens 506A, 506B, 506D, and 506N are arranged within concentric region 520 of guide catheter 504 such that all the lumens are spaced apart from each other. The average spacing of lumens 506A, 506B, 506D, and 506N from 506C may be any of the following values or in a range of any two of the following values: 0.30, 0.35, 0.40, 0.45, and 0.5 millimeters. The average spacing between lumens 506A, 506B, 506D, and 506N may be any of the following values or in a range of any two of the following values: 0.80, 0.85, 0.90, 0.95, and 1.0 millimeters. In one or more embodiments, the average radial spacing (e.g., the spacing of lumens 506A, 506B, 506D, and 506N from 406C) is less than the average concentric spacing (e.g., the spacing between lumens 506A, 506B, 506D, and 506N).

As shown in FIGS. 5A and 5C, lumen 506C is located along the longitudinal axis of tapered portion 514 of guide catheter 504 and lumens 506A, 506B, 506D, and 506N are angled within concentric region 522 of guide catheter 504 at distal end 518 thereof such that all the lumens are spaced apart from each other. The average spacing of lumens 506A, 506B, 506D, and 506N from 506C may be any of the following values or in a range of any two of the following values: 1.6, 1.7, 1.8, 1.9, and 2.0 millimeters. The average spacing between lumens 506A, 506B, 506D, and 506N may be any of the following values or in a range of any two of the following values: 2.0, 2.1, 2.2, 2.3, and 2.4 millimeters. In one or more embodiments, the average radial spacing (e.g., the spacing of lumens 506A, 506B, 506D, and 506N from 406C) is less than the average concentric spacing (e.g., the spacing between lumens 506A, 506B, 506D, and 506N). The spacing of lumens are configured to maintain the spacing of guidewires 502A, 502B, 502C, 502D, and 502N within tapered portion 514 of guide catheter 504 and to aid in coverage of a cross section area corresponding to the aortic leaflets such that at least one of the guidewires crosses the aortic leaflets.

While five (5) guidewire/lumen combinations are shown in FIGS. 5A and 5B, more than five (5) guidewire/lumen combinations may be carried within guide catheter 504 (e.g., 6, 7, 8, 9, 10, 11, and 12 guidewire/lumen combinations). Guide catheter 504 may accommodate any of the following number of guidewires or any range including any two of the following number of guidewires: 4, 5, 6, 7, 8, 9, 10, 11, and 12. Increasing the number of guidewires may increase the likelihood that one or more of the guidewires cross the aortic leaflets. In one or more embodiments, one or more of the guidewires have a diameter of 0.89 millimeters. One or more of the guidewires may have any of the following diameters or a range of any two of the following diameters: 0.75, 0.80, 0.85, 0.89, 0.95, and 1.0 millimeters.

In one or more embodiments, multiple guidewires 502A, 502B, 502C, 502D, and 502N are loosely connected through the apertures of a control knob such that a force exerted on control knob in a longitudinal direction of guide catheter 504 advances the multiple guidewires in the longitudinal direction while an opposing force (c.g., a reaction force or a pulling force) may retract one or more of the multiple guidewires. A reaction force may be exerted on one or more of the guidewires when one or more of the guidewires contacts the aortic wall or an aortic leaflet, thereby causing the one or more guidewires to retract (c.g., push backwards) due to the reaction force. A pulling force may be exerted by a clinician by grasping a proximal portion of one or more of the guidewires. The pulling force may be used on the guidewires that do not cross the aortic leaflets to remove those guidewires before subsequent procedural steps (e.g., inserting a guide catheter over one of the crossing guidewires and exchanging it for a pigtail and/or J-tip wire). In one or more embodiments, guide catheter 504 may be removed from the patient's vasculature before these subsequent steps.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, case of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An aortic leaflet crossing system comprising:
a guide catheter defining a guide lumen therein, the guide catheter having a proximal end defining a proximal opening and a distal end defining a distal opening;
a plurality of guidewires extending into the proximal opening and through the guide lumen and out of the distal opening; and
a control device configured to advance the plurality of guidewires such that at least one of the guidewires crosses aortic leaflets of an aortic valve and to retract guidewires in the plurality of guidewires that do not cross the aortic leaflets.

2. The aortic leaflet crossing system of claim 1, wherein the guide catheter has a longitudinal axis, and the control device is configured to translate axially along the longitudinal axis to advance the plurality of guidewires.

3. The aortic leaflet crossing system of claim 1, wherein the control device defines a plurality of apertures, and the plurality of guidewires is connected to the control device through the plurality of apertures.

4. The aortic leaflet crossing system of claim 3, wherein the control device has a proximal surface and a distal surface, and the plurality of apertures extend through the proximal surface and the distal surface.

5. The aortic leaflet crossing system of claim 3, wherein a first group of the plurality of apertures is arranged in a first concentric region of the control device and a second group of the plurality of apertures are arranged in a second concentric region of the control device.

6. The aortic leaflet crossing system of claim 5, wherein the second concentric region has a second diameter greater than a first diameter of the first concentric region, and the second group contains more guidewires than the first group.

7. The aortic leaflet crossing system of claim 1, wherein the plurality of guidewires includes 15 or more guidewires.

8. An aortic leaflet crossing system comprising:
a guide catheter defining a plurality of guide lumens having a plurality of proximal ends defining a plurality of proximal openings and a plurality of distal ends defining a plurality of distal openings;
a plurality of guidewires extending into the plurality of proximal openings and through the plurality of guide lumens and out of the plurality of distal openings; and
a control device configured to advance the plurality of guidewires such that at least one of the guidewires crosses aortic leaflets of an aortic valve and to retract guidewires in the plurality of guidewires that do not cross the aortic leaflets.

9. The aortic leaflet crossing system of claim 8, wherein one of the plurality of guide lumens is aligned with a longitudinal axis of the guide catheter.

10. The aortic leaflet crossing system of claim 8, wherein a first group of the plurality of guide lumens is arranged in a first concentric region of the guide catheter.

11. The aortic leaflet crossing system of claim 8, wherein the guide catheter includes a cylindrical portion and a tapered portion distally extending from the cylindrical portion, and the tapered portion tapers outward from a proximal end to a distal end.

12. The aortic leaflet crossing system of claim 11, wherein the plurality of guide lumens in the cylindrical portion has a first average spacing, the plurality of guide lumens in the tapered portion has a second average spacing, and the second average spacing is greater than the first average spacing.

13. The aortic leaflet crossing system of claim 11, wherein at least some of the guide lumens are angled relative to a longitudinal axis of the guide catheter.

14. The aortic leaflet crossing system of claim 11, wherein at least one of the guide lumens is aligned with a longitudinal axis of the guide catheter.

15. The aortic leaflet crossing system of claim 8, wherein the plurality of guide lumens includes 5 or more guide lumens, and the plurality of guidewires includes 5 or more guidewires.

16. An aortic leaflet crossing method comprising:
inserting a guide catheter carrying a plurality of guidewires into a vasculature, the guide catheter having a proximal end defining a proximal opening and a distal end defining a distal opening;
delivering the guide catheter to an aorta;
advancing the plurality of guidewires through the guide catheter and out of the distal opening such that at least one of the guidewires crosses aortic leaflets of an aortic valve; and
retracting guidewires in the plurality of guidewires that do not cross aortic leaflets of the aortic valves.

17. The aortic leaflet crossing method of claim 16, wherein the retracted guidewires are obstructed by the aortic leaflets.

18. The aortic leaflet crossing method of claim 16, wherein the retracted guidewires and the guide catheter are removed from the vasculature of the patient.

19. The aortic leaflet crossing method of claim 18 further comprising advancing a second guide catheter over one of the at least one guidewire crossing the aortic leaflets of the aortic valve.

20. The aortic leaflet crossing method of claim 16, wherein the retracted guidewires are withdrawn through a control device configured to advance and to retract the guidewires.
